(19) United States
(12) Patent Application Publication
(10) Pub. No.: US 2009/0221590 A1
(43) Pub. Date: Sep. 3, 2009

(54) SALT OF CD 80 ANTAGONIST

(75) Inventor: Ian Richard Matthews, Oxfordshire (GB)

Correspondence Address: BANNER & WITCOFF, LTD., 1100 13th STREET, N.W., SUITE 1200, WASHINGTON, DC, 20005-4051, US

(73) Assignee: MEDIGENE LIMITED, ABINGDON, OXFORDSHIRE (GB)

(21) Appl. No.: 12/279,898

(22) PCT Filed: Feb. 19, 2007
(86) PCT No.: PCT/GB07/00550

§ 371 Date: Mar. 5, 2009

(30) Foreign Application Priority Data
Feb. 22, 2006 (GB) 0603522.4

(51) Int. Cl. A61K 31/5025; C07D 487/04

(52) U.S. Cl. 514/248; 544/234

(57) ABSTRACT

The choline salt of the CD80 antagonist compound 4-(6-fluoro-3-oxo-1,3-dihydro-pyrazolo[4,3-c]cinnolin-2-yl)-N-(2,2-difluoro-ethyl)-benzamide (I) has good aqueous solubility and is therefore convenient for pharmaceutical use.

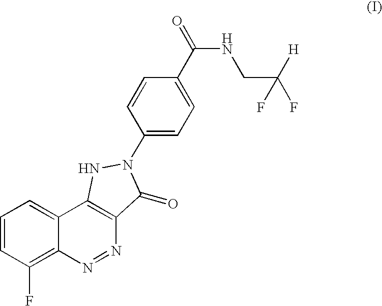

The present invention relates to the choline salt of the CD80 antagonist compound 4-(6-fluoro-3-oxo-1,3-dihydro-pyrazolo[4,3-c]cinnolin-2-yl)-N-(2,2-difluoro-ethyl)-benzamide.

### BACKGROUND TO THE INVENTION

International patent application No. WO 2004/08101 relates to a class of compounds including (inter alia) the compound 4-(6-fluoro-3-oxo-1,3-dihydro-pyrazolo[4,3-c]cinnolin-2-yl)-N-(2,2-difluoro-ethyl)-benzamide, having the structural formula (A):

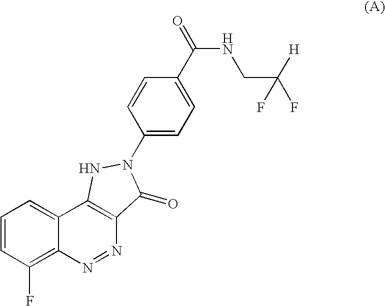

The compounds disclosed in WO 2004/08101 are CD80 antagonists, capable of inhibiting the interactions between CD80 and CD28, and therefore useful for immuno-inhibition, for example in the treatment of rheumatoid arthritis. The compound (A), in the form of the free acid, is poorly soluble in water. In general good water solubility is a desirable characteristic in a compound intended for oral administration, or parenteral or topical administration in a water based carrier, as a pharmaceutical product. WO 2004/08101 also refers to salts of the compound class of which compound (A) is a member. However, not all salts of the compound have sufficiently improved aqueous solubility over the free acid to be convenient orally administrable, or aqueous solution-based, forms of the compound.

### BRIEF DESCRIPTION OF THE INVENTION

This invention is based on the finding that the choline salt of compound (A) has the desired good aqueous solubility.

### DETAILED DESCRIPTION OF THE INVENTION

According to the present invention, there is provided the choline salt of 4-(6-fluoro-3-oxo-1,3-dihydro-pyrazolo[4,3-c]cin nolin-2-yl)-N-(2,2-difluoro-ethyl)-benzamide of formula (A) above.

Orally administrable pharmaceutical compositions comprising the said choline salt and at least one pharmaceutically acceptable carrier also form an aspect of the invention.

Injectable aqueous solutions of the said choline salt, and topically applicable aqueous based liquid or cream compositions containing the said choline salt form another aspect of the invention.

The orally administrable compositions may be in the form of tablets, capsules, pellets, powders, granules, lozenges, liquid or gel preparations. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The active ingredient may also be administered parenterally in a sterile medium, by injection or infusion. Depending on the vehicle and concentration used, the drug can either be suspended or dissolved in the vehicle. Advantageously, adjuvants such as a local anaesthetic, preservative and buffering agents can be dissolved in the vehicle.

The preparation of, and aqueous solubility of, the choline salt of the invention are further described in the following Example.

### Example

4-(6-Fluoro-3-oxo-1,3-dihydro-pyrazolo[4,3-c]cinnolin-2-yl)-N-(2,2-difluoro-ethyl)-benzamide was prepared and its choline (ie (2-hydroxy-ethyl)-trimethyl-ammonium) salt formed, as follows:

### Preparation of N-(2,2-Difluoro-ethyl)-4-(6-fluoro-3-oxo-1,3-dihydro-pyrazolo[4,3-c]cinnolin-2-yl)-benzamide

A round bottom flask equipped with a magnetic stirrer, reflux condenser and gas bubbler was charged with 4-(6-Fluoro-3-oxo-1,3-dihydro-pyrazolo[4,3-c]cinnolin-2-yl)-benzoic acid (12.9 g) prepared as in Example 5 of WO 2004/08101. Thionyl chloride (65 ml) was added slowly. A nitrogen atmosphere was applied and the mixture was heated to reflux. Upon heating gas evolution was observed, the gas was trapped in a scrubber. When the gas evolution had ceased (after approx. 2 h) the mixture had changed colour from an orange-red suspension to a dark red suspension and was cooled to room temperature. Excess thionyl chloride was removed under vacuum and the obtained red solid was azeotroped with toluene (50 ml). A dark red solid was obtained and taken up in anhydrous DMA (65 ml) to give a dark red solution. Diisopropyl ethyl amine (12.9 g, 17.4 ml) was mixed with 2,2-difluoroaminoethane (3.24 g, 2.76 ml) and added drop-wise to the solution over 4-5 minutes. An exothermic was observed. The mixture was stirred at room temperature.

The reaction mixture was quenched with 0.5 M HCl (150 ml) to give a dark red suspension. The solids were collected by filtration and washed with some water. The solid was triturated with methanol (250 ml) for approx. 1 hour at room temperature, filtered and washed with methanol. The solid was then triturated with acetone (250 ml) for approx. 1 hour at room temperature, filtered and washed with acetone. The solid was finally triturated with ethyl acetate (250 ml) for approx. 45 minutes, filtered and washed with ethyl acetate.

The product (approx. 8 g) was taken up in approx. 10 ml of DMA to give a thick dark red solution. Methanol (150 ml) and acetone (150 ml) were added and the precipitate collected by filtration.

LC-MS: One peak of required product. QC analysis of this material showed a purity of 91.6%.

### Preparation of 2-[4-(2,2-Difluoro-ethylcarbamoyl)-phenyl]-6-fluoro-2H-pyrazolo[4,3-c]cinnolin-3-olate (2-hydroxy-ethyl)-trimethyl-ammonium

A round bottom flask fitted with a magnetic stirrer was charged with N-(2,2-Difluoro-ethyl)-4-(6-fluoro-3-oxo-1,3-dihydro-pyrazolo[4,3-c]cinnolin-2-yl)-benzamide (2.50 g). The solid was suspended in methanol (25 ml) and choline hydroxide (782 mg, 1.74 ml 45% w/w in methanol) was added drop-wise. Upon addition a clear dark brown solution was formed. The solution was stirred for 2 hours at room temperature. The reaction mixture was concentrated under vacuum to give a dark brown oil. Ethyl acetate (100 ml) was added and a two-phase system was obtained. After sonication the brown oil became thick and started to solidify. The solid was broken up and isopropyl alcohol (30 ml) was added and the mixture was heated to 65-70° C. A suspension was obtained. The mixture was cooled to room temperature and diluted with ethyl acetate (approx. 100 ml). The solids were collected by filtration and washed with ethyl acetate (50 ml). The solid was dried under vacuum.

LC-MS: isolated solid; one main peak [M+H]+ 388, purity 95%.

The solid salt was a brown coarse powder, 2.26 g, 4.61 mmol, 71% Melting point: 172-180° C., decomposition.

The product was further purified by recrystallisation, as follows: The salt was mixed with ethanol and heated to reflux, stirred for 20 minutes, and filtered hot. The filtered ethanol solution was heated to reflux, then cooled to about 5 deg C. and stirred for one hour. The solution was then filtered, washed with ethanol and heptane, and dried to constant weight.

The solubility of the choline salt of N-(2,2-difluoro-ethyl)-4-(6-fluoro-3-oxo-1,3-dihydro-pyrazolo[4,3-c]cinnolin-2-yl)-benzamide in water and ethanol was tested in the following assay. The solubilities of the non-salt form (free acid) N-(2,2-difluoro-ethyl)-4-(6-fluoro-3-oxo-1,3-dihydro-pyrazolo[4,3-c]cin nolin-2-yl)-benzamide and a range of other salts thereof were also tested in the same assay:

### Solubility Assay

10 mg of the test compound was weighed into a vial, and 1 ml of solvent was added. If the sample dissolved immediately, as evidenced by a formation of a clear solution, more of the test material was added and the vial sonicated for 15 mins. This procedure was repeated until no more test compound could be dissolved, as evidenced by formation of a suspension. The vial containing the suspension was then was sonicated for 15 mins, and placed on a shaker at 25 deg C./60% relative humidity for 24 hours. On removal from the shaker, the sample was centrifuged and the supernatant analysed by HPLC. The results are given in Table I.

| | Solubility (mg/ml) in | Solubility (mg/ml) in |
|---|---|---|
| Test Salt | water | ethanol |
| None (free acid) | <0.5 | 2.1 |
| Choline | >51 | >21 |
| Potassium | <5 | <5 |
| Sodium | <5 | 7.1 |
| Magnesium | <5 | <5 |
| Lysine | <5 | <5 |
| Arginine | <5 | <5 |

1. The choline salt of 4-(6-fluoro-3-oxo-1,3-dihydro-pyrazolo[4,3-c]cinnolin-2-yl)-N-(2,2-difluoro-ethyl)-benzamide of formula (A):

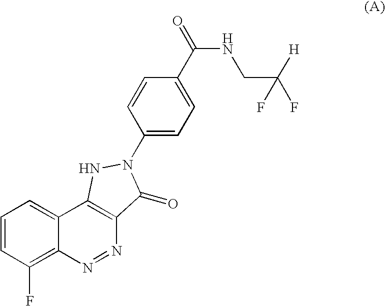

2. An orally administrable pharmaceutical composition containing the choline salt claimed in claim 1 and at least one pharmaceutically acceptable carrier.

3. An injectable aqueous solution of the said choline salt claimed in claim 1.

4. A topically applicable aqueous based liquid or cream composition containing the choline salt claimed in claim 1.
